(19) United States
(12) Patent Application Publication
(10) Pub. No.: US 2018/0055968 A1
(43) Pub. Date: Mar. 1, 2018

(54) TUNABLE SURFACE WETTABILITY OF FIBER BASED MATERIALS

(71) Applicants: Kimberly-Clark Worldwide, Inc., Neenah, WI (US); CLEMSON UNIVERSITY RESEARCH FOUNDATION, Clemson, SC (US)

(72) Inventors: Keyur Desai, Greenville, WI (US); Donald Eugene Waldroup, Roswell, GA (US); Andrew Thomas Baker, Norcross, GA (US); Yuriy Galabura, Appleton, WI (US); Fehime Vatansever, Neenah, WI (US); Igor Luzinov, Seneca, NC (US); Ruslan Burtovyy, Anderson, SC (US); Michael James Seeber, New York, NY (US); James Matthew Giammarco, Sharon Hill, PA (US)

(21) Appl. No.: 15/561,588

(22) PCT Filed: Oct. 28, 2015
(86) PCT No.: PCT/US15/57716

§ 371 Date: Sep. 26, 2017

(51) Int. Cl. A61L 15/24; A61F 13/513; A61F 13/15; A61L 15/26; A61L 15/44; A61L 15/22; A61L 15/60

(52) U.S. Cl. CPC A61L 15/24; A61F 13/513; A61F 13/15642; A61L 15/26; A61L 2420/06; A61L 15/225; A61L 15/60; A61L 2300/606; A61L 2420/02; A61L 15/44

(57) ABSTRACT

A method for making a permanently wettable material is disclosed. The method includes selecting a plurality of non-polar polymer fibers (12) wherein each fiber has a surface (16), depositing a hydrophilic polymer mixture (14) on the non-polar polymer fiber surface to form a shell. The hydrophilic polymer mixture (14) includes a cross-linkable and graftable epoxy-containing polymer, such as, poly(glycidyl methacrylate-co-ethylene glycol methacrylate) copolymer (PGMA-co-POEGMA), a high weight average molecular weight polyethylene glycol (PEG), and a surfactant. A permanently wettable material is also disclosed that includes a non-polar polymer-based web (10) having fibers (12) with a surface (16). A hydrophilic polymer mixture (14) forms a shell on the non-polar polymer fiber surface (16). The hydrophilic polymer mixture (14) includes a poly(glycidyl methacrylate-co-ethylene glycol methacrylate) copolymer (PGMA-co-POEGMA), a high weight average molecular weight polyethylene glycol (PEG), and a surfactant.

### TECHNICAL FIELD

A method of modifying fibers that forms a functional, wettable polymer shell around the individual fibers is described.

### BACKGROUND OF THE DISCLOSURE

Polymers are used extensively to make a variety of products which include blown and cast films, extruded sheets, injection molded articles, foams, blow molded articles, extruded pipe, monofilaments, fibers and nonwoven webs. Polymers can be categorized in several ways, such as for example, they may be hydrophilic or hydrophobic. Some polymers, such as polyolefins, are naturally hydrophobic, which is disadvantageous in certain applications.

There are a number of uses for polymers where their hydrophobic nature either limits their usefulness or requires some effort to modify the surface characteristics of the articles made therefrom. By way of example, polyolefins, such as polyethylene and polypropylene, are used to manufacture polymeric fabrics that can be employed in the construction of disposable absorbent articles. Such polymeric fabrics are often nonwoven webs prepared by, for example, such processes as melt-blowing, carding, coforming and spunbonding. Frequently, such polymeric fabrics need to be wettable by water or aqueous-based fluids. Wettability can be obtained by spraying or otherwise coating, such as for example, surface treating or topically treating, the fabric with a surfactant solution during or after its formation, and then drying the web.

Disposable absorbent articles, especially personal care absorbent articles, such as diapers, training pants, sanitary napkins, incontinence products, and so forth, typically include at least one nonwoven polymeric fabric. For example, many personal care absorbent articles can include a liner that separates the wearer's skin from the materials that constitute the absorbent core, such as cellulose fluff and/or superabsorbent material. In many instances, the liner material is a polypropylene-based spunbond web that has been surface treated with surfactants. Since polyolefin nonwoven fabrics and other types of polymeric fabrics are normally hydrophobic and water-repellent, surfactant treatment increases the wettability of the web surface. The surfactant treated liner allows urine or other aqueous-based fluids to readily flow through the liner material and to be absorbed by the absorbent core. A major drawback with the surfactant based approach to make a hydrophilic polypropylene substrate is that after the first insult of body fluid to the substrate, the body fluid washes the surfactant off of the substrate surface and the substrate loses its ability to allow subsequent fluid insults to flow through the substrate.

For example, diapers can experience three or more insults of urine that may occur over the time that the diaper is worn, which may be a period of hours and on average can be 4 hours. The first insult of urine onto the liner can wash the surfactant off of the liner. Over time, this wetted, surfactant-free area on the liner will be desorbed by the underlying layers and once dry, this area becomes hydrophobic due to the natural properties of the polypropylene. Subsequent fluid insults then move along the liner surface that is against the wearer's skin until the fluid finds a place where the liner surface is wettable and the fluid can flow through the liner to be absorbed by the absorbent core. Each subsequent insult must spread further to find a wettable location on the liner. The risk of urine leakage from the diaper increases as the distance that the fluid must travel and absorption time increase.

Accordingly, there remains a need to permanently impart hydrophilicity or wettability to fibers of nonwoven fabrics.

### SUMMARY OF THE DISCLOSURE

In one aspect, the disclosure includes a method for making a permanently wettable material. The method includes selecting a plurality of non-polar polymer fibers. Each non-polar polymer fiber has a surface. The method also includes depositing a hydrophilic polymer mixture on the non-polar polymer fiber surface to form a shell.

In another aspect, the disclosure includes a method for making a permanently wettable nonwoven material. The method includes selecting a polypropylene-based web having fibers. Each fiber has a surface. The methods also includes functionalizing the polypropylene-based web fiber surface by oxidizing the fiber surface with plasma treatment or corona discharge. The method further includes depositing the polypropylene-based web fiber surface with a hydrophilic polymer mixture to form a shell. The hydrophilic polymer mixture includes a poly(glycidyl methacrylate-co-ethylene glycol methacrylate) copolymer (PGMA-co-POEGMA), a high weight average molecular weight polyethylene glycol (PEG), and a surfactant.

In a further aspect, the disclosure includes a permanently wettable material. The permanently wettable material includes a non-polar polymer-based web having fibers. Each fiber has a surface. A hydrophilic polymer mixture forms a shell on the non-polar polymer fiber surface. The hydrophilic polymer mixture includes a poly(glycidyl methacrylate-co-ethylene glycol methacrylate) copolymer (PGMA-co-POEGMA), a high weight average molecular weight polyethylene glycol (PEG), and a surfactant.

### BRIEF DESCRIPTION OF DRAWINGS

FIG. 1A representatively illustrates a nonwoven fibrous substrate according the disclosure.

FIG. 1B representatively illustrates an expanded view of fibers of the substrate shown in FIG. 1A with a shell completely on the fiber surface.

FIG. 1C representatively illustrates a cross section of the shell on the fiber surface taken at line 1C-1C of FIG. 1B.

FIG. 2A representatively illustrates a nonwoven fibrous substrate according the disclosure.

FIG. 2B representatively illustrates an expanded view of fibers of the substrate shown in FIG. 2A with a shell on portions of the fiber surface.

FIG. 2C representatively illustrates a cross section of the shell on the fiber surface taken at line 2C-2C of FIG. 2B.

FIG. 2D representatively illustrates a cross section of an alternate configuration of the shell on the fiber surface taken at line 2C-2C of FIG. 2B.

FIG. 3A representatively illustrates an incontinence pad.

FIG. 3B representatively illustrates a schematic cross section of the incontinence pad taken at line 3B-3B of FIG. 3A.

FIG. 4A representatively illustrates a perspective view of a diaper.

FIG. 4B representatively illustrates an exploded view of the diaper of FIG. 4A.

### DETAILED DESCRIPTION OF THE DISCLOSURE

The term “carded web” refers herein to a web containing natural or synthetic staple length fibers typically having fiber lengths less than about 100 mm. Bales of staple fibers can undergo an opening process to separate the fibers which are then sent to a carding process which separates and combs the fibers to align them in the machine direction after which the fibers are deposited onto a moving wire for further processing. The web is usually subjected to some type of bonding process such as thermal bonding using heat and/or pressure, ultrasonic bonding, or may be subject to adhesive processes to bind the fibers together. The carded web may be subjected to fluid entangling, such as hydroentangling, to further intertwine the fibers and thereby improve the integrity of the carded web.

The term “coform” refers herein to a blend of meltblown fibers and absorbent fibers such as cellulosic fibers that can be formed by air forming a meltblown polymer material while simultaneously blowing air-suspended fibers into the stream of meltblown fibers. The meltblown fibers and absorbent fibers are collected on a forming surface, such as provided by a belt. Two U.S. patents describing coform materials are U.S. Pat. No. 5,100,324 to Anderson et al. and U.S. Pat. No. 5,350,624 to Georger et al., both of which are incorporated in their entirety in a manner consistent herewith.

The term “durable” refers herein to a hydrophilic polymeric material on a fiber or other fibrous substrate such that the treated fiber or fibrous substrate remains wettable after multiple exposures to an aqueous-based fluids, such as water, saline, urine, and other body fluids. One procedure for evaluating the durability of the fibrous web is to determine the wettability after being washed with aqueous liquid and with hydrostatic pressure testing. The fibrous web is considered to be permanently wettable by maintaining a hdyrostatic pressure of 2.0 mbars or less, according to test method AATCC 127-2003 with the modifications specified herein, after the web has undergone a wash period of at least 10 minutes with de-ionized water and subsequent drying of the web at room temperature (22 degrees Celsius and 55 percent humidity) prior to being tested.

The term “fibrous hydrophobic polymer substrate” refers herein to include any shaped article, provided it is porous and composed, in whole or in part, of a hydrophobic polymer. For example, the substrate may be a sheet-like material that may be a fibrous web, such as a woven or nonwoven fabric or web. The substrate also may be a hydrophobic polymer fiber or hydrophobic polymer fibers which have been formed into a fibrous web. A fibrous nonwoven web, for example, can be, but is not limited to, a meltblown web, a spunbonded web, a carded web, a wet-laid web or an airlaid web. The substrate also may be a laminate of two or more layers of a sheet-like material. For example, the layers may be independently selected from the group consisting of meltblown webs and spunbonded webs. However, other sheet-like materials such as films or foams may be used in addition to, or instead of, meltblown and spunbonded webs. In addition, the layers of the laminate may be prepared from the same hydrophobic polymer or different hydrophobic polymers. The fibrous hydrophobic substrate includes hydrophobic nonwovens that may also include natural fibers.

The term “hydrophilic” refers herein to having a strong affinity for aqueous-based fluids and/or to be wetted by aqueous-based fluids. Generally, when the water contact angle to a solid surface is smaller than 90 degrees, the solid surface is considered to by hydrophilic.

The term “hydrophilic polymer” refers herein to any polymer that promotes wetting by aqueous-based fluids.

The term “hydrophobic” refers herein to the ability to repel aqueous-based fluids. Generally, when the water contact angle to a solid surface is larger than 90 degrees. The solid surface is considered to be hydrophobic.

The term “hydrophobic polymer” refers herein to any polymer that is resistant to wetting, or not readily wet, by aqueous-based fluids.

The term “meltblown” refers herein to fibers formed by extruding a molten, thermoplastic material through a plurality of fine, usually circular, die capillaries as molten threads or filaments into converging high velocity gas (e.g., air) streams, generally heated, which attenuate the filaments of molten thermoplastic material to reduce their diameters. Thereafter, the meltblown fibers are carried by the high velocity gas stream and are deposited on a collecting surface or support to form a web of randomly dispersed meltblown fibers. Such a process is disclosed, for example in U.S. Pat. No. 3,849,241 to Butin et al. which is incorporated herein by reference in its entirety in a manner consistent herewith.

The term “non-polar polymer” refers herein to a polymer or mixture of polymers that does not have a positive or negative charge, or a dipole.

The terms “nonwoven”, “nonwoven fabric” and “nonwoven web” refer herein to materials and webs of material that are formed without the aid of a textile weaving or knitting process. For example, nonwoven materials, fabrics or webs have been formed from many processes such as, for example, meltblowing processes, spunbonding processes, air laying processes, coform processes, and bonded carded web processes, and can include webs formed of combinations thereof.

The term “personal care absorbent articles” refers herein to a disposable absorbent article that may be placed against or in proximity to the body (i.e., contiguous with the body) of a wearer to absorb and contain various liquid and solid waste discharged from the body. Non-limiting examples of articles that may be placed against or in proximity to the body include, for example, diapers, diaper pants, training pants, disposable swimwear, absorbent underpants, adult incontinence products including garments and insert pads, bed pads, feminine hygiene pads or liners, digital tampons, sweat absorbing pads, shoe pads, helmet liners, wipes, tissues, towels, napkins, underpads and the like.

The term “polar polymer” refers herein to a polymer or mixture of polymers that includes a positive or negative charge. Some examples of polar polymers are polyamide, polycarbonate, poly(methyl methacrylate), and acrylonitrile butadiene styrene.

The term “polyolefin” is used herein to mean a polymer prepared by the addition polymerization of one or more unsaturated monomers which contain only carbon and hydrogen atoms. The polyolefin may contain additives as is known in the art. For example, the polyolefin may contain pigments, opacifiers, fillers, delustrants, antioxidants, antistatic agents, stabilizers, oxygen scavengers, and so forth.

The terms “spunbonded fibers” and “spunbond” refer herein to small diameter fibers which are formed by extruding molten thermoplastic material as filaments from a plurality of fine, usually circular-capillaries of a spinneret with the diameter of the extruded filaments then being rapidly reduced to fibers as by, for example, in U.S. Pat. No. 4,340,563 to Appel et al.; U.S. Pat. No. 3,692,618 to Dorschner et al.; U.S. Pat. No. 3,802,817 to Matsuki et al.; U.S. Pat. Nos. 3,338,992 and 3,341,394 to Kinney; U.S. Pat. No. 3,502,763 to Hartman; and U.S. Pat. No. 3,542,615 to Dobo et al., the contents of which are incorporated herein by reference in their entirety in a manner consistent herewith.

The term “thermoplastic” refers herein to a material which softens and which can be shaped when exposed to heat and which substantially returns to a non-softened condition when cooled.

The terms “woven”, “woven fabric” or “woven web” refer herein to materials and webs of material that are formed with the aid of a textile weaving or knitting process.

The present disclosure relates to a method of modifying a non-polar polymer fiber or a plurality of non-polar polymer fibers that results in a functional shell around the fiber(s) such that the fiber(s) are permanently wettable. The terms “wetting” or “wettable” refer herein to the ability of a solid substrate to reduce the surface tension of a liquid that is in contact with the substrate to such extent that the liquid spreads over the surface of the substrate and completely wets it. The shell includes a cross-linkable and graftable epoxy-containing polymer. The epoxy-containing polymer is deposited onto the non-polar polymer fiber and can be grafted onto the non-polar polymer fiber surface and/or cross-linked to itself to form the functional shell. The functional shell can be further modified by grafting low molecular weight substances, biomolecules, and/or polymers to the shell.

The fibers or plurality of fibers suitable for the disclosed method include those made from non-polar polymers. Non-polar polymers are naturally hydrophobic since they inherently do not have a charge or dipole. As such, the non-polar polymers are not wettable with water or aqueous-based fluids as water molecules are polar and are not attracted to the non-polar polymer. Non-polar polymers are frequently used in personal care absorbent articles and thus need to be wettable when used for particular purposes.

Non-polar polymers that are hydrophobic can include polyolefins, such as, but not limited to, polyethylene, polypropylene, poly(1-butene), poly(2-butene), poly(1-pentene), poly(2-pentene), poly(3-methyl-1-pentene), poly(4-methyl-1-pentene), poly(isobutene), poly(isoprene), ethylene-propylene copolymers, ethylene-propylene-hexadiene copolymers, and ethylene-vinyl acetate copolymers; styrene polymers, such as poly(styrene), poly(2-methylstyrene), styrene-acrylonitrile copolymers having less than about 20 mole-percent acrylonitrile, and styrene-2,2,3,3,-tetrafluoropropyl methacrylate copolymers; halogenated hydrocarbon polymers, such as poly(chlorotrifluoroethylene), chlorotrifluoroethylene-tetrafluoroethylene copolymers, poly(hexafluoropropylene), poly(tetrafluoroethylene), tetrafluoroethylene-ethylene copolymers, poly(trlfluoroethylene), poly(vinyl fluoride), and poly(vinylidene fluoride); vinyl polymers, such as poly(vinyl butyrate), poly(vinyldecanoate), poly(vinyl dodecanoate), poly(vinyl hexadecanoate), poly(vinyl hexanoate), poly(vinyl propionate), poly(vinyl octanoate), poly(heptafluoroisopropoxyethylene), poly(heptafluoroisopropoxypropylene), and poly(methacrylonitrile); acrylic polymers, such as poly(n-butylacetate), poly(ethyl acrylate), poly[(1-chlorodifluoromethyl) tetrafluoroethyl acrylate], poly[di(chlorofluoromethyl)fluoromethyl acrylate], poly(1, 1-dihydroheptafluorobutylacrylate), poly(1, 1-dihydropentafluoroisopropyl acrylate), poly(1, 1-dihydropentadecafluorooctyl acrylate), poly(heptafluoroisopropyl acrylate), poly[5-heptafluoroisopropoxy) pentyl acrylate], poly[11-(heptafluoroisopropoxy)undecyl acrylate], poly[2-(heptafluoropropoxy)ethyl acrylate], and poly(nonafluoroisobutyl acrylate); methacrylic polymers, such as poly(benzyl methacrylate), poly(n-butyl methacrylate), poly(isobutyl methacrylate), poly(t-butyl methacrylate), poly(t-butylaminoethyl methacrylate), poly(dodecyl methacrylate), poly(ethyl methacrylate), poly(2-ethylhexyl methacrylate), poly(n-hexyl methacrylate), poly(phenyl methacrylate), poly(n-propyl methacrylate), poly(octadecyl methacrylate), poly(1, 1-dihydropentadecafluorooctyl methacrylate), poly(heptafluoroisopropyl methacrylate), poly (heptadecafluorooctyl methacrylate), poly(1-hydrotetrafluoroethyl methacrylate), poly(1, 1-dihydrotetrafluoropropyl methacrylate), poly(1-hydrohexafluoroisopropyl methacrylate), and poly)t-nonafluorobutyl methacrylate); and polyesters, such a poly(ethylene terephthalate) and poly(butylene terephthalate). Additionally, blends of two or more polyolefins and random and block copolymers prepared from two or more different unsaturated monomers can also be non-polar.

While polar, naturally hydrophilic polymers can include, poly(acrylic acid), poly(methacrylic acid), or poly(2-acrylamido-2-methylpropanesulfonic acid), polyacrylamide, polymethacrylamide, poly(hydroxyethylacrylate), poly(dimethylaminoalkyl (meth)-acrylate), poly(ethoxylated (meth)-acrylates), poly(dimethylaminopropyl acrylamide), or poly acrylamidopropyltrimethyl ammonium chloride and their copolymers and blends; poly(N-isopropylacryl amide), poly(2-oxazoline), polyethyleneimine, polyethylene glycol, polyethylene oxide, poly(vinyl alcohol), poly(vinylpirrolidone) and their copolymers; polyelectrolytes, such as, poly(styrenesulfonate), polyacrylamide based polyelectrolytes, poly(acrylic acid) sodium, potassium or ammonium salts, poly(allylamine hydrochloride), poly(diallyldimethylammonium chloride), polyvinyl acid and their copolymers and blends.

The non-polar polymers can be formed into non-polar polymer fibers wherein each fiber includes a surface. Subsequently, a plurality of non-polar polymer fibers can be formed into nonwoven or woven webs or fabrics. As an example, a nonwoven web can be made from non-polar, hydrophobic synthetic fibers, such as polyolefin fibers. The polyolefin fibers that form the web may include polyethylene and/or polypropylene fibers and fibers produced from compositions and blends that include a polyethylene and/or a polypropylene polymer. The hydrophobic polymer fibers and/or nonwoven substrate generally may be prepared by any known means. For example, the fibers can be prepared by a melt-extrusion process and formed into a fibrous web, such as a nonwoven web. The melt-extrusion process refers herein to forming a nonwoven web in which the polymer is melt-extruded to form a plurality of fibers, the fibers are formed into a web, usually on a porous support. Melt-extrusion processes can included, but are not limited to melt-blowing, coforming, and spunbonding.

Other methods for preparing nonwoven webs may include air laying, wet laying, and carding. In some cases, it may be desirable or necessary to stabilize the nonwoven web by known means, such as thermal point bonding, through-air bonding, and hydroentangling. In addition to nonwoven webs, the hydrophobic polymer fibers may be in the form of continuous filaments or staple fibers, as well as woven or knitted fabrics or webs prepared from such continuous filaments or staple fibers. Furthermore, the nonwoven web may include bicomponent or other multicomponent fibers. Exemplary multicomponent nonwoven webs are described in U.S. Pat. No. 5,382,400 issued to Pike et al., U.S. patent application Ser. No. 10/037,467 entitled “High Loft Low Density Nonwoven Webs of Crimped Filaments and Methods of Making Same” and U.S. patent application Ser. No. 10/136,702 entitled “Methods For Making Nonwoven Materials On A Surface Having Surface Features And Nonwoven Materials Having Surface Features” which are hereby incorporated by reference herein in their entirety. Sheath/core bicomponent fibers where the sheath is a polyolefin such as polyethylene or polypropylene and the core is polyester such as poly(ethylene terephthalate) or poly(butylene terephthalate) can also be used to produce carded webs or spunbonded webs.

In some applications where a permanently wettable, non-polar polymer web is desired, such as, for example in personal care absorbent articles, a hydrophilic polymer mixture is grafted onto the non-polar polymer fiber surface. The terms “graft”, “grafting” or “grafted” refer herein to a process wherein one material can be affixed to another material through such processes as, but not limited to, absorption, adsorption, bond formation (e.g., ionic, nonionic, etc.), polymerization, or any other suitable method to affix one material to another from the melt, gas phase, or liquid phase. For example, the surface of a polypropylene-based substrate is very inert due to the absence of any reactive functionalities in the polypropylene microstructure. Thus, grafting of other polymers that possess reactive functionalities to the polypropylene demands additional treatment of the substrate to create reactive sites that allow grafting to occur. The treatment includes oxidizing the non-polar polymer fiber surface through chemical oxidation, flame treatment, UV radiation, and more commonly, plasma treatment or corona discharge. It should be understood that the web may undergo a cleaning process prior to the oxidation treatment to remove dust, dirt particles, and/or any surface finish from the spunbonding, or fiber-forming process, as is known in the art. A cleaning process may include soaking the web in a solvent bath. For example, a cleaning process for a polypropylene spunbond web may involve soaking the web in two different solvents for 30 minutes each. The web is air-dried at room temperature after the cleaning process.

The oxidation treatment introduces reactive functional groups on the non-polar polymer fiber surface. For example, reactive functional groups that are introduced through oxidation of a polypropylene-based substrate, include hydroxyl, carbonyl, and carboxy groups that are capable of reacting with other complimentary, functional groups of the polymers being grafted to the substrate surface.

A hydrophilic polymer mixture can be grafted onto the oxidized, non-polar polymer fiber surface to form a shell on the fiber surface. The hydrophilic polymer mixture includes an epoxy-containing polymer, a viscosity enhancer, and a surfactant.

Epoxy-containing polymers can be cross-linked or otherwise stabilized to form an anchoring layer of the shell on the non-polar polymer fiber surface. It should be noted that since epoxy-containing polymers are highly reactive under a wide variety of conditions, the anchoring layer may not be required, or may not be formed in aspects of the present disclosure. In addition, the particular bond formed between the non-polar polymer fiber surface and the epoxy groups can depend upon the functionality on the non-polar polymer fiber surface and as such, the epoxy-containing polymer may be bound via covalent bonds, hydrogen bonds, ionic bonds, or any other strong or weak bond. In aspects that include an anchoring layer, the anchoring layer can be formed on the non-polar polymer fiber surface to a depth of at least about 0.5 nanometers, or between about 1 and about 10 nanometers, or at least about 100 nanometers. Two U.S. patents describing anchoring layers are U.S. Pat. No. 7,026,014 and U.S. Pat. No. 7,261,938 to Luzinov et al., both of which are incorporated in their entirety in a manner consistent herewith.

While the epoxy groups of the epoxy-containing polymer can be utilized to firmly bind the epoxy-containing polymer to the non-polar polymer fiber surface via cross-linking, a number of the epoxy groups can remain intact even if an anchoring layer is formed. These remaining, reactive epoxy groups can then be utilized for subsequent binding of additional materials to the non-polar polymer fiber surface. Optionally, the epoxy-containing polymers may be pre-functionalized or otherwise preprocessed according to any desired methodology as is known in the art to provide a specific epoxy-containing polymer. For example, the epoxy-containing polymer can include backbone material or side chain functionality so as to interact in a specific way with the non-polar polymer fiber surface or with materials that can be subsequently grafted to the remaining intact epoxy groups.

Suitable epoxy-containing polymers can include, although are not limited to, epoxidized polybutadiene, epoxidized polyisoprene, poly(glycidyl methacrylate) (PGMA), and poly(glycidyl methacrylate-co-ethylene glycol methacrylate) (PGMA-co-POEGMA). In general, any epoxy-containing homopolymer or copolymer possessing about ten or more oxyrane rings per polymer can be utilized. In some aspects, the polymer can include greater epoxy functionality, such as, for example, the polymer can include one or more epoxy groups on each repeating unit of the polymer. In other aspects, the polymer can be a block, graft, alternating, or random copolymer, in which at least one of the monomers found in the copolymer includes one or more epoxy functionalities on each monomer unit, while the other monomer(s) carry no epoxy functionality. In an aspect where PGMA-co-POEGMA is grafted onto the non-polar polymer fiber surface of a polypropylene fibrous nonwoven substrate, the PGMA is the anchoring portion which helps attach the hydrophilic portion, POEGMA, to the polypropylene, non-polar polymer fiber surface.

The hydrophilic polymer mixture includes a viscosity enhancer that increases the viscosity of the mixture. For example, polyethylene oxide or a high weight average molecular weight polyethylene glycol (PEG) may be suitable. Any PEG of weight average molecular weight between about 100,000 Da and about 1,000,000 Da may be used. Based on the weight average molecular weight of the PEG that is used and the desired concentration, for the range given, a PEG solution can range from about 5 percent to about 0.5 percent by weight, respectively, for example.

The hydrophilic polymer mixture additionally includes a surfactant that makes the non-polar polymer fiber surface wettable such that the epoxy-containing polymer can contact and subsequently attach to the non-polar polymer fiber surface. Any surfactant may be used, including, ionic surfactants, such as cationic or anionic, or neutral surfactants, such as non-ionic or zwitterionic. Neutral surfactants are preferred for personal care products and hygienic products that touch the skin of a user.

Suitable cationic surfactants can include pH-dependent primary, secondary, or tertiary amines, octenidine dihydrochloride, or permanently charged quaternary ammonium cations, such as, for example: 1) alkyltrimethylammonium salts including cetyl trimethylammonium bromide, hexadecyl trimethyl ammonium bromide, and cetyl trimethylammonium chloride, 2) cetylpyridinium chloride, 3) benzalkonium chloride, 4) benzethonium chloride, 5) 5-Bromo-5-nitro-1,3-dioxane, 6) dimethyldioctadecylammonium chloride, 7) cetrimonium bromide, 8) dioctadecyldimethylammonium bromide.

Suitable anionic surfactants may include: 1) those that contain anionic functional groups, such as sulfate, sulfonate, phosphate, and carboxylates, 2) prominent alkyl sulfates including ammonium lauryl sulfate, sodium lauryl sulfate, and the related alkyl-ether sulfates, 3) sodium laureth sulfate, also known as sodium lauryl ether sulfate, 4) sodium myreth sulfate, 5) docusates including dioctyl sodium sulfosuccinate, perfluorooctanesulfonate, perfluorobutanesulfonate, linear alkylbenzene sulfonates, 6) alkyl-aryl ether phosphates, and 7) alkyl ether phosphates. Carboxylates are also a suitable category of anionic surfactants and can include: 1) alkyl carboxylates, such as sodium stearate, 2) sodium lauroyl sarcosinate, and 3) carboxylate-based fluorosurfactants, such as perfluorononanoate or perfluorooctanoate.

Suitable non-ionic surfactants can include: 1) fatty alcohols, such as, cetyl alcohol, stearyl alcohol, and cetostearyl alcohol, and oleyl alcohol, 2) polyoxyethylene glycol alkyl ethers, 3) octaethylene glycol monododecyl ether, 4) pentaethylene glycol monododecyl ether, 5) polyoxypropylene glycol alkyl ethers, 6) glucoside alkyl ethers, 7) decyl glucoside, 8) lauryl glucoside, 9) octyl glucoside, 10) polyoxyethylene glycol octylphenol ethers, 11) Triton X-100, 12) polyoxyethylene glycol alkylphenol ethers, 13) Nonoxynol-9, 14) glycerol alkyl esters, such as glyceryl laurate, 15) polyoxyethylene glycol sorbitan alkyl esters, such as polysorbate, 16) sorbitan alkyl esters, such as spans, 17) cocamide MEA, 18) cocamide DEA, 19) dodecyldimethylamine oxide, 20) block copolymers of polyethylene glycol and polypropylene glycol, such as poloxamers, as for example, LUTROL F127, available from BASF Corporation North America, Florham Park, N.J., USA, or PLURONIC F-127, available from Sigma Aldrich, St. Louis, Mo., USA, and 21) polyethoxylated tallow amine (POEA).

Suitable zwitterionic surfactants can include: 1) sulfonates, such as in CHAPS (3-[(3-Cholamidopropyl)dimethylammonio]-1-propanesulfonate), 2) sultaines such as cocamidopropyl hydroxysultaine, 3) betaines, such as cocamidopropyl betaine, and 4) phosphates suchas lecithin.

Suitable surfactants for use with personal care absorbent articles can include those from the PLURONIC line, such as, PLURONIC F-127 and F-68. Additionally, poloxamers, such as 1) 101, 105, 108, 122, 123, 124, 181, 182, 183, 184, 185, 188, 212, 215, 217, 231, 234, 235, 237, 238, 282, 284, 288, 331, 333, 334, 335, 338, 401, 402, 403, and 407; 2) Poloxalene; 3) KOLLIPHOR P 407; 4) SYNPERONIC F 108; and 5) SYNPERONIC PE P105, SYNPERONIC PE/P84 (all available from Sigma Aldrich, St. Louis, Mo., USA) may also be suitable.

The surfactant level can range on a weight to volume basis, from about 0.005 percent weight/volume to about 2.0 weight/volume percent of the hydrophilic polymer mixture. The surfactant level may vary depending on the surfactant type that is chosen and on the surfactant's critical micelle concentration.

The hydrophilic polymer mixture can be grafted to the non-polar polymer fiber surface. The hydrophilic polymer mixture must first be deposited onto the non-polar polymer fiber surface according to any suitable coating process. Suitable coating methods can include roll-coating, printing, dipping, immersing, soaking, spraying, or by any other suitable application means. For example, the hydrophilic polymer mixture can be deposited on the surface of the non-polar fibers by a dip-coating process utilizing a solution that contains from about 0.02 percent to about 1.0 percent of the hydrophilic polymer, by weight, in an organic-based or water-based solvent. In some aspects, less dilute solutions of the polymer may be utilized. In another aspect, the hydrophilic polymer mixture can be applied in a more controlled deposition process in an effort to apply the hydrophilic polymer mixture heterogeneously to the non-polar polymer fiber surface.

The amount of coverage of the hydrophilic polymer mixture on the fiber surface can be varied such that the fiber surface does not need to be completely covered with the hydrophilic polymer mixture.

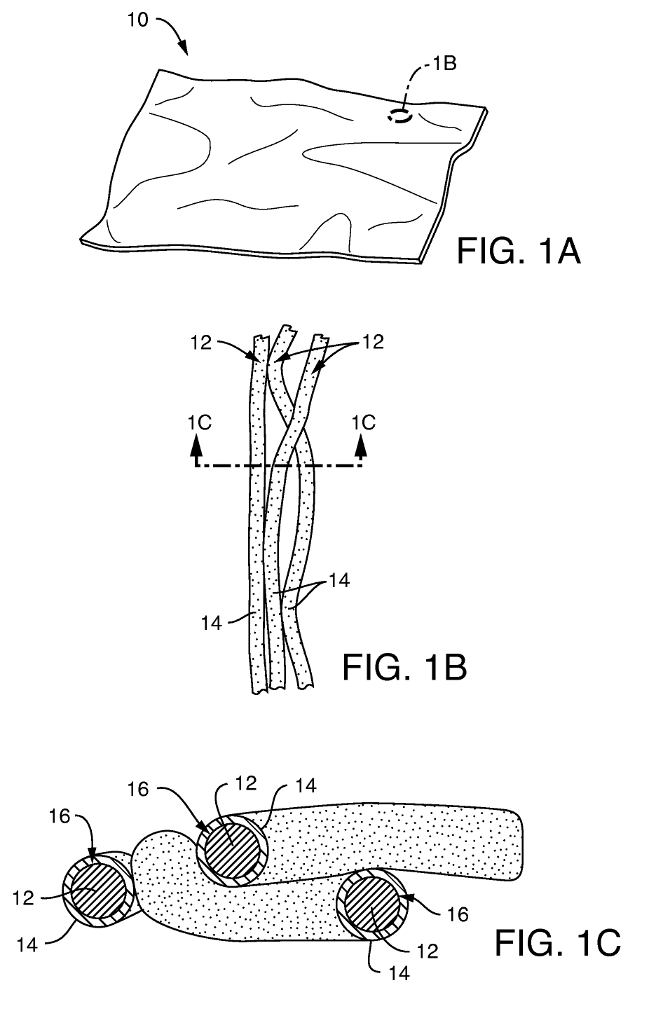

Referring to FIGS. 1A-1C, a nonwoven fibrous substrate 10 that will be completely wettable according to the present disclosure is described. The wettable, nonwoven fibrous substrate 10 includes non-polar polymer fibers 12 having a surface 16. A hydrophilic polymer mixture 14 is coated onto the non-polar polymer fibers 12. FIG. 1B is an expanded view of a few non-polar polymer fibers 12 of the nonwoven fibrous substrate 10 shown in FIG. 1A. In this aspect, the hydrophilic polymer mixture 14 may be coated onto to all of the non-polar polymer fibers 12 at the non-polar polymer fiber surface 16 as shown in FIG. 1C. The nonwoven fibrous substrate 10 is completely wettable as the non-polar polymer fiber surface 16 is completely coated with the hydrophilic polymer mixture 14.

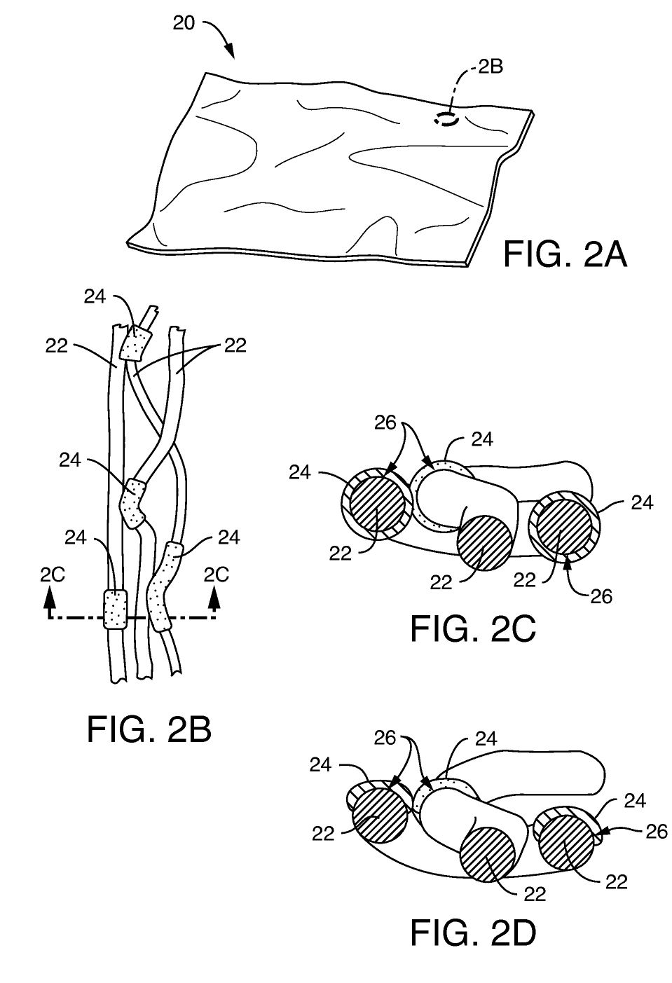

In another aspect, referring to FIGS. 2A-2D, a nonwoven fibrous substrate 20 that will be semi-wettable according to the present disclosure is described. The nonwoven fibrous substrate 20 includes non-polar polymer fibers 22 having a surface 26. A hydrophilic polymer mixture 24 is coated onto the non-polar polymer fibers 22. FIG. 2B is an expanded view of a few non-polar polymer fibers 22 of the nonwoven fibrous substrate 20 shown in FIG. 2A. The hydrophilic polymer mixture 24 may be coated onto to a portion of the non-polar polymer fibers 22 around the circumference of the non-polar polymer fiber surface 26 as shown in FIG. 2C. In yet another aspect, the hydrophilic polymer mixture 24 may be coated onto the non-polar polymer fibers 22 at portions of the circumference of the non-polar polymer fiber surface 26 as shown in FIG. 2D. The nonwoven fibrous substrate 20 is semi-wettable as the non-polar polymer fiber surface 26 is not completely coated with the hydrophilic polymer mixture 24.

The hydrophilic polymer mixture 14, 24 is then annealed to the non-polar polymer fiber surface 16, 26. Annealing can occur over a temperature range of from about room temperature (22 degrees Celsius and 55 percent humidity) to about 120 degrees Celsius. In an aspect, annealing may take place at 120 degrees Celsius for 2 hours.

The hydrophilic polymer mixture 14, 24 forms a shell on the non-polar polymer fiber surface 16, 26 first by grafting to the non-polar polymer fiber surface 16, 26 and then by cross-linking on to itself. The grafting and subsequently, the cross-linking of the hydrophilic polymer mixture 14, 24 occurs as a chain reaction once the epoxy rings of the epoxy-containing polymer are opened. An unexpected technical effect includes the ability of the synthesized epoxy-containing polymer to crosslink on itself during annealing that can also lead to the formation of a permanent shell on the non-polar polymer fiber surface 16, 26. As a result, the need to oxidize the non-polar polymer fiber surface 16, 26 may not be necessary in all circumstances.

The shell may now be further modified by grafting materials to the intact epoxy groups that remain after the hydrophilic polymer mixture 14, 24 has been applied to the non-polar polymer fiber surface 16, 26. Suitable materials may include 1) a macromolecule that may possess any of several different possible functional groups, such as, carboxy, anhydride, amino and/or hydroxy groups, 2) biomolecules, such as proteins, polysaccharides, or antibacterial agents, 3) low molecular weight substances, and/or 4) polymers that have carboxyl, hydroxyl, mercapto, amine, and/or anhydride groups.

The subject application discloses a method of making permanently wettable material. A selecting step involves selecting a plurality of non-polar polymer fibers 12, 22 wherein each fiber 12, 22 has a surface 16, 26. The plurality of non-polar polymer fibers 12, 22 may be a woven or nonwoven fibrous substrate 10, 20. The non-polar polymer fibers 12, 22 may be subjected to a cleaning process.

A functionalizing step involves creating reactive sites on the non-polar polymer fibers 12, 22 by oxidizing the non-polar polymer fiber surface 16, 26. The oxidation can occur through such methods as plasma treatment or corona discharge. For example, general plasma process parameters for oxygen plasma treatment involve time and power where the treatment time may range from about 30 seconds to about 30 minutes depending on the power setting and the substrate.

A deposition step involves forming a shell on the non-polar polymer fiber surface 16, 26 by coating the non-polar polymer fibers 12, 22 with a hydrophilic polymer mixture 14, 24. The coating method may be any coating method that is known in the art, such as for example, dip-coating or spraying. The coating method may allow the hydrophilic polymer mixture 14, 24 to graft to the non-polar polymer fiber surface 16, 26. The hydrophilic polymer mixture 14, 24 includes a hydrophilic polymer that is a cross-linkable and graftable epoxy-containing polymer, a viscosity enhancer, and a surfactant.

An annealing step involves treatment with temperature to dry the hydrophilic polymer mixture 14, 24 on the non-polar polymer fiber surface 16, 26. The annealing step conditions are dependent upon the deposition step process conditions, the substrate and the solvent that was used. In some aspects, an annealing step may not be necessary as the hydrophilic polymer mixture 14, 24 may dry at room temperature and as such, grafting may commence immediately.

The non-polar polymer fibers 12, 22 may be rinsed in de-ionized water (DIW) after the annealing step for as little at 10 minutes or for a longer time period, such as, for example, 8 hours, or even longer. It has been found that the durability of the shell on the non-polar polymer fiber surface 16, 26 was the same whether the web underwent a 10 minute or an 8 hour DIW rinse. The DIW rinse is to simulate the durability of the shell in use in personal care absorbent articles as it is known that the users of personal care absorbent articles may wear the articles for a short time period that includes at least one urine insult, such as 10 minutes, or for a longer time period, such as overnight, or for an approximate 8 hour time period. The web can be air-dried following the DIW rinse.

The method can further include the step of grafting a low molecular weight substance, a biomolecule, or a polymer other than the hydrophilic polymer used in the hydrophilic polymer mixture 14, 24 that forms the shell. Suitable polymers include the presence of complementary functional groups able to react with the epoxy functionalities of the hydrophilic polymer in the hydrophilic polymer mixture 14, 24. Complementary reactive functionalities include, but are not limited to, hydroxyl, carboxyl, mercapto, or amino groups.

In an exemplary aspect, a non-polar polymer fibrous web that is a 18 gsm polypropylene spunbond was selected. The web was cleaned by soaking the web in reagent alcohol and then in methyl-ethyl ketone (MEK), for 30 minutes in each solvent; each solvent was replaced with fresh solvent after 15 minutes. The web was allowed to air dry at room temperature. The web was then surface oxidized with the use of a basic plasma cleaner, model PDC-32G (available from Harrick Plasma, Ithaca, N.Y., USA), set at high power for 5 minutes. The plasma-treated polypropylene spunbond web was then coated by soaking the web for 5 minutes in a hydrophilic polymer mixture that included PGMA-co-POEGMA, high molecular weight polyethylene glycol (PEG), and PLURONIC F-127 surfactant to form a shell on the non-polar polymer fiber surfaces 16, 26.

The PGMA-co-POEGMA is synthesized according to any desired methodology as is known in the art such that the composition of the copolymer is 47 mole percent of glycidyl methacrylate (GMA) and 53 mole percent of oligoethyleneglycol (OEGMA) methacrylate. The mole ratio is 9:10 GMA to OEGMA respectively (based on Nuclear Magnetic Resonance analysis). The copolymer was synthesized by free radical solution polymerization approach.

The hydrophilic polymer mixture was prepared by forming a solution of PGMA-co-POEGMA by taking 1.25 ml of a 4 percent stock solution in MEK and removing the MEK to obtain approximately 0.05 g of the polymer. The polymer was then dissolved in 10 ml of a 0.1 percent g/g PLURONIC F-127 surfactant solution in DIW with 2 ml of a 0.6 percent g/ml high molecular weight PEG solution in DIW for stability. The concentration of the polymer in solution is approximately 0.42 percent g/ml. The web was dried at 80 degrees Celsius for 5 minutes and the hydrophilic polymer mixture was annealed at 120 degrees Celsius for 2 hours. One sample of the resulting web was rinsed with DIW for 10 minutes and another for 8 hours; each was subsequently allowed to air dry at room temperature. The web samples were maintained in a horizontal position, while not under tension, such as through clamping, during the drying process.

Wettability of the modified substrates of the disclosure were measured using a hydrostatic pressure test, AATCC 127-2003 with the modifications disclosed herein. Table 1 includes hydrostatic pressures for the following 18 gsm polypropylene spunbond substrate (available from Kimberly-Clark Corporation, Dallas, Tex., USA) codes: A) untreated, B) treated with a 0.5 percent weight/volume solution of PLURONIC F-127 and air dried at room temperature, C) the surfactant treated substrate (code B) washed with DIW for 10 minutes and air dried at room temperature, D) the exemplary aspect, and E) the exemplary aspect washed with DIW for 10 minutes and air dried at room temperature. Codes C and E were washed with water to simulate urine insults and moisture on a liner material of a personal care absorbent article. For example, it is known that a substantial amount of surfactant can be washed off with an initial urine insult onto a diaper liner. Each subsequent urine insult can additionally remove more surfactant such that a 10 minute DIW wash was incorporated into the test protocol to simulate actual use.

It has been found that unmodified polypropylene substrates (code A) that are naturally hydrophobic and have no wettability have hydrostatic pressures of at least 10 millibars (mbars). The substrates treated for wettability, either with surfactant or the hydrophilic polymer mixture of the present disclosure, codes B and D, have initial, hydrostatic pressures that range from about 1.0 to about 2.0 mbars. After the surfactant treated code is washed with water for 10 minutes (code C), it can be seen that much of the surfactant has been washed away from the substrate as indicated by the increase in hydrostatic pressure to about 6 mbars to about 7 mbars. After the exemplary aspect has been washed with water for 10 minutes (code E), the wettability is maintained as indicated by the hydrostatic pressure remaining constant from about 1.5 mbars to about 2.0 mbars.

In another aspect, the exemplary aspect was washed with DIW for 8 hours and produced the same results as those reported for Code E in Table 1. It should be understood that while an 8 hour DIW wash may far exceed simulation of actual product use, an 8 hour DIW wash further demonstrates the durability of the present disclosure, specifically for the exemplary aspect.

TABLE 1
Hydrostatic Pressures for Polypropylene Spunbond Webs

| | | | CODE C - | | |
|---|---|---|---|---|---|
| | | | Surfactant | | |
| | CODE A - | CODE B - | Treated | | CODE E - |
| | Unmod- | Surfactant | Polypro- | CODE D - | Exemplary |
| | ified | Treated | pylene: | Exemplary | Aspect: |
| | Polypro- | Polypro- | Washed-10 | Aspect: | Washed-10 |
| | pylene | pylene | minutes | Initial | minutes |
| P_{water}, | 9.0-13.0 | 1.0-1.5 | 5.5-7.0 | 1.5-2.0 | 1.5-2.0 |
| mbars | | | | | |

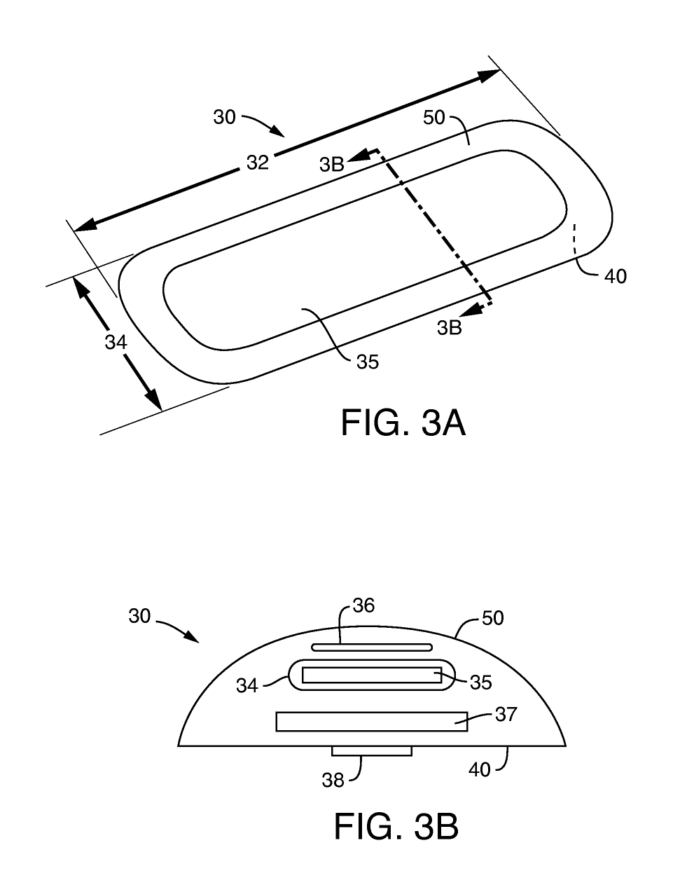

Referring to FIGS. 3A and 3B, an example of a personal care absorbent article including wettable materials according to the present disclosure is an incontinence pad 30 having a length 32 and a width 34. The incontinence pad 30 includes a liquid pervious liner 50, which is designed to allow fluids, such as, for example, urine, blood, or runny fecal matter to quickly pass therethrough. The liner 50 may be made from nonwoven webs, such as, for example, polypropylene spunbonded webs or multi-component bonded carded webs.

Referring now to FIG. 3B, in some aspects, between the barrier sheet 40 and the liner 50, is an absorbent core 35 and optionally, a number of layers for different purposes. Other optional layers may include an acquisition layer 36, a distribution layer 37, and a tissue wrap 34. The acquisition layer 36 and the distribution layer 37 may be made from nonwoven substrates, such as a polypropylene bonded carded web, an airlaid web composed of natural and synthetic fibers, or may be a layer composed of a meltblown or spunbond web of synthetic fibers, for example, polyolefin fibers. The bonded carded web may, for example, be a thermally bonded web which is bonded using low melt binder fibers, powder or adhesive. The webs can include a mixture of different fibers. Although the acquisition layer 36 and the distribution layer 37 may be made up of one or more layers of materials, each of these components will be referred to as one layer for descriptive purposes in this disclosure.

The acquisition layer 36 may be positioned beneath the liner 50 and acts as a reservoir to accept large surges of liquid and slowly release them to, for example, absorbent core 35. In some aspects, wherein the absorbent core 35 further includes superabsorbent particles, the tissue wrap 34 can surround the absorbent core 35 and contain the superabsorbent particles. In yet other aspects, the distribution layer 37 may be positioned beneath the tissue wrap 34 wherein the distribution layer 37 is designed to distribute bodily exudates that are not readily absorbed upon initial contact with the absorbent core 35.

The liner 50 is at the top and an acquisition layer 36 is positioned below the liner 50. Below the acquisition layer 36 is the absorbent core 35 surrounded by tissue wrap 34. Distribution layer 37 is positioned below the tissue wrapped absorbent core 35. The barrier sheet 40 is below the distribution layer 37. Many products also have an adhesive strip 38 placed on the outer surface of the barrier sheet 40 to help hold the product in place during use by adhering it to the user's underclothes.

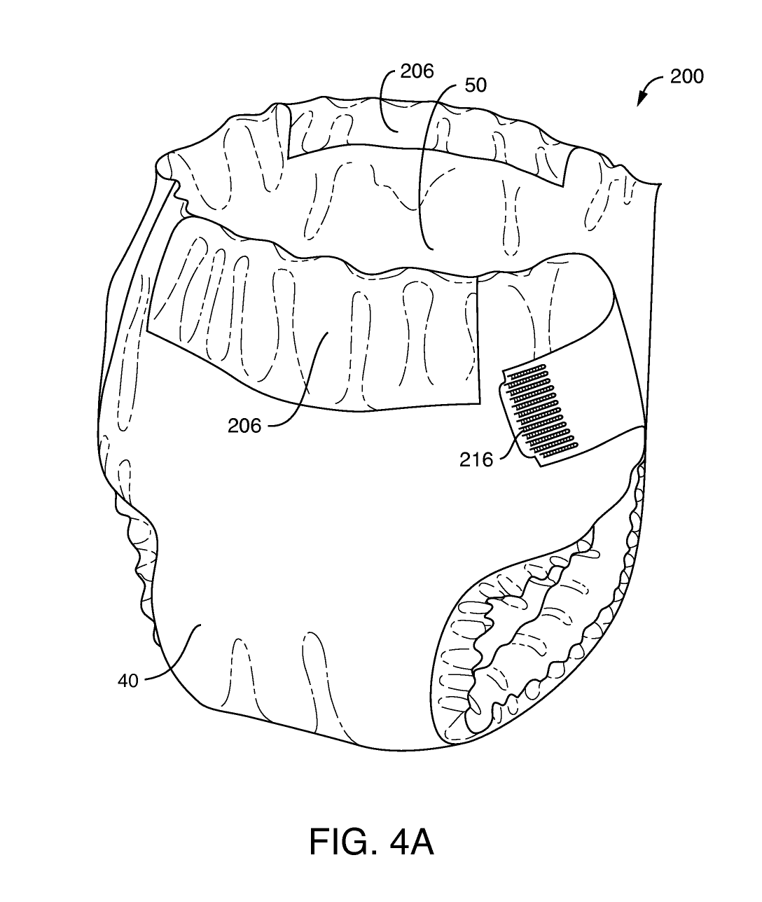

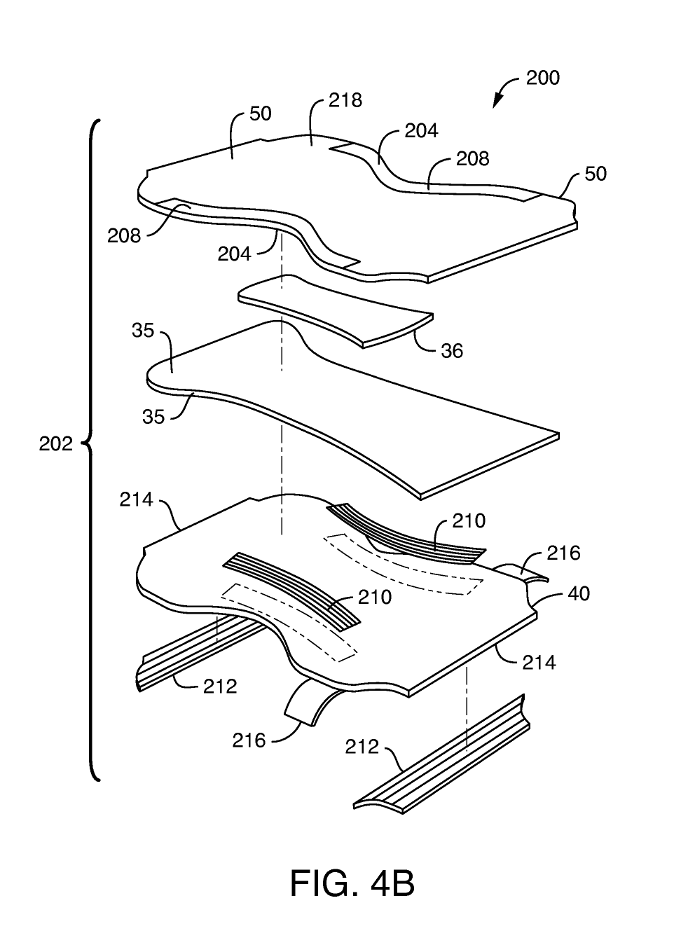

Referring to FIGS. 4A and 4B, in yet another aspect, the permanently wettable material according to the disclosure is included in a diaper 200. The diaper 200 includes a chassis 202 formed by various components, including a barrier sheet 40, a liquid pervious liner 50, an absorbent core 35, and an acquisition layer 36. Besides the above-mentioned components, the diaper 200 may also contain various other components as is known in the art, such as, for example, tissue wrap 34 and distribution layer 37 (not illustrated). Likewise, one or more of the layers referred to in FIG. 4B may also be eliminated in certain exemplary embodiments. The diaper 200 is shown as having an hourglass shape in an unfastened configuration in FIG. 4B. However, other shapes may be utilized, such as a generally rectangular shape, T-shape, or I-shape. In some embodiments, the diaper 200 may also include a pair of side panels, or ears, (not shown) that extend from the side edges 204 of the diaper 200 into one of the waist regions 206. The side panels may be integrally formed with a selected diaper component. For example, the side panels may be integrally formed with the barrier sheet 40.

The diaper 200 may also include a pair of containment flaps 208 that are configured to provide a barrier and to contain the lateral flow of body exudates. The containment flaps 208 may be located along the laterally opposed side edges 204 on an outwardly facing surface 218 of the liner 50 adjacent the side edges 204. The containment flaps 208 may extend longitudinally along the entire length of the diaper 200, or may only extend partially along the length of the diaper 200.

To provide improved fit and to help reduce leakage of body exudates, the diaper 200 may be elasticized with suitable elastic members. For example, the diaper 200 may include leg elastics 210 constructed to operably tension the side margins of the diaper 200 to provide elasticized leg bands which can closely fit around the legs of the wearer to reduce leakage and provide improved comfort and appearance. Waist elastics 212 may also be employed to elasticize the end margins 214 of the diaper 200 to provide elasticized waistbands. The waist elastics 212 are configured to provide a resilient, comfortably close fit around the waist of the wearer.

The diaper 200 may also include one or more fasteners 216. For example, two flexible fasteners 216 on opposite side edges 204 of waist regions 206 to create a waist opening and a pair of leg openings about the wearer. The shape of the fasteners 216 may generally vary, but may include, for instance, generally rectangular shapes, square shapes, circular shapes, triangular shapes, oval shapes, linear shapes, and so forth. The fasteners may include, for instance, a hook-and-loop material, buttons, pins, snaps, adhesive tape fasteners, cohesives, fabric-and-loop fasteners, etc. In one particular embodiment, each fastener 216 includes a separate piece of hook material affixed to the inside surface of a flexible backing.

The various regions and/or components of the diaper 200 may be assembled together using any known attachment mechanism, such as adhesive, ultrasonic, thermal bonds, etc. Suitable adhesives may include, for instance, hot melt adhesives, pressure-sensitive adhesives, and so forth. When utilized, the adhesive may be applied as a uniform layer, a patterned layer, a sprayed pattern, or any of separate lines, swirls or dots. In the illustrated embodiment, for example, the barrier sheet 40 and liner 50 are assembled to each other and to the absorbent core 35 using an adhesive. Similarly, other diaper 200 components, such as the leg elastic members 210, waist elastic members 212 and fasteners 216, may also be assembled into the diaper 200 using any attachment mechanism.

The absorbent core 35 includes liquid absorbent material such as cellulosic fibers and superabsorbent particles to absorb urine and fecal matter. The absorbent core is located between the laterally opposed side edges 204 and the longitudinally opposed end margins 214.

Regardless of the particular form of the personal care absorbent article, it can be recognized that various components made of non-polar polymers, for example, the liner 50, the acquisition layer 36, and the distribution layer 37, need to be wettable to enhance the urine or aqueous fluid distribution and performance of the personal care absorbent article.

### EXAMPLES

Table 2 includes the raw data for the values shown in Table 1.

| | CODE A - | CODE B - | CODE C - | CODE D - | CODE E - |
|---|---|---|---|---|---|
| Sample | (P_{water}, | (P_{water}, | (P_{water}, | (P_{water}, | (P_{water}, |
| Number | mbars) | mbars) | mbars) | mbars) | mbars) |
| 1 | 9.0 | 1.0 | 6.5 | 1.5 | 1.5 |
| 2 | 10.0 | 1.0 | 6.5 | 1.5 | 1.5 |
| 3 | 12.0 | 1.5 | 5.5 | 2.0 | 2.0 |
| 4 | 13.0 | 1.5 | 7.0 | 2.0 | 2.0 |
| 5 | 12.5 | | | | |
| 6 | 13.0 | | | | |
| Average | 11.58 | 1.25 | 6.38 | 1.75 | 1.75 |
| Standard | 1.54 | 0.25 | 0.54 | 0.25 | 0.25 |
| Deviation | | | | | |

### Test Methods

Test method AATCC 127-2003, Option 2 Tester (American Association of Textile Chemists and Colorists, Research Triangle Park, N.C., USA) was used with the following modification, for each sample tested, to minimize lateral water leakage:
a. Cut a piece of 15.0 cm×15.0 cm REYNOLDS WRAP heavy duty aluminum foil.
b. Cut out a 3.5 cm diameter circle of foil from the center of the aluminum foil square from step a. to create a hole.
c. Glue a 4.0 cm×4.0 cm piece of web substrate material to the aluminum foil such that the web material is centered over the hole providing a test area of the web material with a diameter of 3.5 cm. LOCTITE Epoxy 5 Minute Instant Mix adhesive was used to glue the substrate to the aluminum foil.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

1. A method for making a permanently wettable material comprising:
selecting a plurality of non-polar polymer fibers wherein each fiber has a surface; and
depositing a hydrophilic polymer mixture on the non-polar polymer fiber surface to form a shell,
wherein the hydrophilic polymer mixture includes a cross-linkable and graftable epoxy-containing polymer.

2. The method of claim 1, further comprising functionalizing the non-polar polymer fibers by creating reactive sites on the fiber surface prior to the depositing step through oxidation.

3. The method of claim 2, wherein the oxidation is completed through plasma treatment or corona discharge.

4. The method of claim 1, further comprising annealing the hydrophilic polymer mixture after deposition on to the non-polar polymer fiber surface.

5. (canceled)

6. The method of claim 1, wherein the cross-linkable and graftable epoxy-containing polymer is poly(glycidyl methacrylate-co-ethylene glycol methacrylate) copolymer (PGMA-co-POEGMA).

7. The method of claim 1, wherein the hydrophilic polymer mixture further comprises a viscosity enhancer.

8. The method of claim 7, wherein the viscosity enhancer is a high molecular weight polyethylene glycol (PEG) that includes a weight average molecular weight between about 100,000 Da and about 1,000,000 Da.

9. The method of claim 1, wherein the hydrophilic polymer mixture further comprises a surfactant.

10. The method of claim 1, further comprising grafting a low molecular weight substance, a bio-molecule, or a polymer to the shell.

11. The method of claim 1, wherein the non-polar polymer fibers are included in a nonwoven web.

12. The method of claim 11, wherein the nonwoven web is a polypropylene-based spunbond.

13. A method for making a permanently wettable nonwoven material comprising: selecting a polypropylene-based web having fibers wherein each fiber has a surface;
functionalizing the polypropylene-based web fiber surface by oxidizing the fiber surface with plasma treatment or corona discharge; and
depositing the polypropylene-based web fiber surface with a hydrophilic polymer mixture to form a shell, wherein the hydrophilic polymer mixture includes a poly(glycidyl methacrylate-co-ethylene glycol methacrylate) copolymer (PGMA-co-POEGMA), a high weight average molecular weight polyethylene glycol (PEG), and a surfactant.

14. The method of claim 13, further comprising grafting a low molecular weight substance, a bio-molecule, or a polymer to the shell.

15. A permanently wettable material comprising:
a non-polar polymer-based web having fibers wherein each fiber has a surface; and
a hydrophilic polymer mixture forming a shell on the non-polar polymer fiber surface, wherein the hydrophilic polymer mixture includes a poly(glycidyl methacrylate-co-ethylene glycol methacrylate) copolymer (PGMA-co-POEGMA), a high weight average molecular weight polyethylene glycol (PEG), and a surfactant.

16. The permanently wettable material of claim 15, wherein the non-polar polymer-based web includes a polyolefin-based spunbond.

17. The permanently wettable material of claim 16, wherein the polyolefin-based spunbond web has been oxidized with plasma treatment or corona discharge.

18. The permanently wettable material of claim 15, wherein the high molecular weight polyethylene glycol is selected from the group consisting of polyethylene oxide and a high molecular weight polyethylene glycol (PEG) that includes a weight average molecular weight between about 100,000 Da and about 1,000,000 Da.

19. The permanently wettable material of claim 15, wherein the surfactant is a neutral surfactant.

20. The permanently wettable material of claim 15, wherein the material exhibits a hydrostatic pressure of less than about 2.0 millibars per test method AATCC-127 with the modifications specified herein, after the web has undergone a wash period of at least 10 minutes with de-ionized water and subsequent drying of the web at room temperature prior to being tested.
